(19) United States
(12) Patent Application Publication
(10) Pub. No.: US 2021/0403995 A1
(43) Pub. Date: Dec. 30, 2021

(54) HIGH-THROUGHPUT METHODS TO CHARACTERIZE PHAGE RECEPTORS AND RATIONAL FORMULATION OF PHAGE COCKTAILS

(71) Applicant: The Regents of the University of California, Oakland, CA (US)

(72) Inventors: Vivek K. Mutalik, Albany, CA (US); Adam P. Arkin, San Francisco, CA (US); Adam M. Deutschbauer, Berkeley, CA (US)

(21) Appl. No.: 17/473,968

(22) Filed: Sep. 13, 2021

Related U.S. Application Data
(63) Continuation of application No. PCT/US20/23010, filed on Mar. 16, 2020.
(60) Provisional application No. 62/818,659, filed on Mar. 14, 2019.

(51) Int. Cl. C12Q 1/6869; C12N 15/10

(52) U.S. Cl. CPC C12Q 1/6869; C12N 15/1082

(57) ABSTRACT

The present invention provides for a method for screening for gene function for a bacteriophage, the method comprising: (1) (a) providing one or more host organism, such as a species or strain, libraries, (b) providing randomly barcoded transposon sequencing (such as RB-TnSeq), and (c) screening for loss-of-function (LOF) mutant phenotypes; or (2) (a) providing one or more DNA barcoded overexpression strain libraries (such as Dub-seq) using DNA of the host organism and/or phage, and (b) screening for gain-of-function (GOF).

### CROSS-REFERENCE TO RELATED APPLICATIONS

The application is a continuation of International PCT Patent Application No. PCT/US20/23010, filed Mar. 16, 2020, which claims priority to U.S. Provisional Patent Application Ser. No. 62/818,659, filed Mar. 14, 2019, all of which are herein incorporated by reference in their entireties.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of production of indigoidine.

### BACKGROUND OF THE INVENTION

There is increasing evidence that the virome—the community of viruses/bacteriophages that interact with microbial communities—is a critical feature of microbial ecology, evolution, virulence, fitness, host physiology and nutrient cycling (Buchan, et al., Nat Rev Microbiol 12, 686-698, 2014; Clemente, et al., Cell 148, 1258-1270, 2012; Philippot, et al., Nat Rev Microbiol 11, 789-799, 2013; hereby incorporated by reference in their entireties). However, despite nearly a century of pioneering molecular work on the mechanisms of a handful of key phage and their hosts, it is only recently that the diversity of phage types, their range of hosts, and their impacts on the activity and dynamics of microbiomes has begun to be studied (Brum et al., Nat Rev Microbiol 13, 147-159, 2015; Roucourt, et al., Environ Microbiol 11, 2789-2805, 2009; Koskella, et al., Viruses 5, 806-823, 2013; hereby incorporated by reference in their entireties). It is now clear that to gain insights into coevolution of bacteria and their associated phages, it is essential to understand their interaction networks, including the mechanisms of phage infection and the breadth of bacterial responses to it. Gaining knowledge of phage-bacteria interactions in general, and the diverse mechanisms of phage resistance in particular, can impact areas as diverse as water quality, food contamination, agricultural yield, and human health (Kutter, E. et al. Phage therapy in clinical practice: treatment of human infections. Curr Pharm Biotechnol 11, 69-86, 2010; Balogh, et al., Curr Pharm Biotechnol 11, 48-57, 2010; Hagens, S. et al., Curr Pharm Biotechnol 11, 58-68, 2010; hereby incorporated by reference in their entireties). For example, because of the apparent ubiquity of lytic phage with high host specificity for nearly any known pathogenic bacterial strain, phages may provide a powerful alternative or adjutant to antibiotic therapies (Nobrega, et al, Trends Microbiol 23, 185-191, 2015; hereby incorporated by reference in its entirety). Development of such therapeutic phage is pressing due to the rise of antibiotic resistance. Thus determining the mechanisms underlying and evolution of phage host range is critical to discovering and developing effective phage treatments for infection (Koskella, et al., Viruses 5, 806-823, 2013; Kortright, et al., Cell Host and microbe, 25, 219, 2019; hereby incorporated by reference in their entireties).

Screening for phage infection or resistance against a panel of bacterial strains is an age-old microbiological scheme still practiced today for characterizing new phage isolates and bacterial strains. These studies generally involve isolation of phage-resistant host mutants (either evolved naturally or created by mutagenesis approaches), and characterization of resistant mutants via cross-infection patterns against a panel of phages using qualitative and phenotypic characterization methods (Dy, et al., Annu Rev Virol 1, 307-331, 2014; Labrie, et al., Nat Rev Microbiol 8, 317-327, 2010; Samson, et al., Nat Rev Microbiol 11, 675-687, 2013; hereby incorporated by reference in their entireties). The best-studied phage/host interaction systems fall into a small handful of fairly related organisms and their double-stranded DNA phages (Diaz-Munoz and Koskella, Adv Appl Microbiol 89, 135-183, 2014; hereby incorporated by reference in its entirety). From these studies, a list of host features such as LPS variants, membrane proteins/channels, and other surface organelles serve the most dominant host-specifying targets for phage (De Smet, et al., Nat Rev Microbiol, 2017; hereby incorporated by reference in its entirety). In turn, for classes of phage like Caudovirales there are specific elements in the tail structures that specifically recognize the appropriate variants of the target host surface. These phage-host interaction studies have generally involved laborious experiments on a single phage and their hosts. Over many years they have revealed, for example, overlapping but distinct mechanisms of host recognition, entry, replication and lysis within the E. coli Type 1-Type 7 (T1 to T7) phages and that resistance to phage can result from a defect at any stage of phage infection (Table 1, Silva et al., FEMS Microbiology letters, 363, 2016; Letarov and Kulikov, Biochemistry (Moscow), 82, 13, 1632-1658, 2017; hereby incorporated by reference in their entireties). Recently, a number of antiphage host mechanisms such as restriction modification, CRISPR-Cas, and BREX systems have been discovered that block phage nucleic acid entry, replication and enhance degradation (De Smet, et al., Nat Rev Microbiol, 2017; Kortright, et al., Cell Host and microbe, 25, 219, 2019; hereby incorporated by reference in their entireties). We do not yet understand the breadth of phage defenses displayed by majority of microbes.

With advent of sequencing technologies, researchers have begun to characterize phage-resistance mechanisms by isolating, and whole genome sequencing a panel of phage resistant mutants (Denes, et al., Appl Environ Microbiol., 81, 4295-4305, 2015; hereby incorporated by reference in its entirety). Though genome sequencing is becoming relatively cheaper, extending whole-genome sequencing to hundreds of phage-resistant mutants to gain insights into all possible resistance mechanisms is currently not an economically viable option. In this context, there have been few attempts to use forward-genetic approaches for studying host factors essential in phage-infection pathways and uncover phage-resistance mechanisms. These loss-of-function genetic screens broadly included use of bacterial saturation mutagenesis library or a library of single gene deletion and have enabled identification of host-factors essential in phage infection, even though applied to individual phage-host combination (Qimron et al., PNAS, 103, 50, 19039-19044, 2006; Maynard et al., PLoS Genet 6, 7, e1001017. 2010; Christen et al, J Mol Biol., 428, 419-430, 2016; Cowley et al., mBio, 9, e00705-18; hereby incorporated by reference in their entireties).

Alternative to LOF genetic screens, which are intuitive in their experimental design for phage resistance studies, GOF screens to study gene dosage effects on phage resistance are not reported widely. Unlike antibiotic resistance studies where overexpression of an efflux pump or increased gene dosage effects is well documented, effect of gene dosage on phage resistance has for the most part not been studied. A recent example of this approach in E coli, where an ASKA library was used to screen host factors that interfere with T7 mutant phage, found that overexpression of rcsA (enhanced colanic acid production) yields resistance to T7 (Qimron et al., PNAS, 103, 50, 19039-19044, 2006; hereby incorporated by reference in its entirety). This suggests that use of GOF libraries to uncover gene dosage effects or system-level genetic barriers on phage growth might yield new mechanisms that LOF screens may not address. However important, currently used genome-wide screening methods using both GOF and LOF libraries to discover phage-host interaction determinants are low throughput and cannot be scaled to assay dozens of phages at different multiplicity of infection for a number of hosts under variable conditions. Such large-scale studies applied to different host-phage combinations have the unique potential to identify commonalities in phage resistance mechanisms and phage specific resistance responses, and these system-level insights will be valuable in understanding ecology of phage resistance and enable us in developing different design strategies in phage therapy application.

### SUMMARY OF THE INVENTION

The present invention provides for a method for screening for gene function for a bacteriophage, the method comprising: (1) (a) providing one or more host organism, such as a species or strain, libraries, (b) providing randomly barcoded transposon sequencing (such as RB-TnSeq), and (c) screening for loss-of-function (LOF) mutant phenotypes; or (2) (a) providing one or more DNA barcoded overexpression strain libraries (such as Dub-seq) using DNA of the host organism and/or phage, and (b) screening for gain-of-function (GOF).

The present invention provides for a method for screening for gene function for a bacteriophage, the method comprising: (a) providing one or more host organism, such as a species or strain, libraries, (b) providing randomly barcoded transposon sequencing (such as RB-TnSeq), and (c) screening for loss-of-function (LOF) mutant phenotypes.

In some embodiments, the providing one or more host organism libraries comprises inserting a barcoded transposon into a host organism, such as using the method taught in Example 1, wherein the host organism(s) can be any host organism, such as any described in Table 1.

The present invention provides for a method for screening for gene function for a bacteriophage, the method comprising: (a) providing one or more DNA barcoded overexpression strain libraries (such as Dub-seq) using DNA of the host organism and/or phage, and (b) screening for gain-of-function (GOF).

In some embodiments, the providing one or more DNA barcoded overexpression strain libraries using DNA of the host organism and/or phage comprises cloning a partial or total host/phage genome DNA fragments into a library of barcoded vector, such as a vector that can stably reside in the host organism, wherein each resulting vector comprises a host/phage genone DNA fragment integrated into the vector, such as using the method taught in Example 1, wherein the host organism(s) can be any host organism, such as any described in Table 1.

In some embodiments, where needed, the providing step comprises end repairing the fragments, phosphoylating the repaired fragments, and ligating the phosphorylated repaired fragments to the vector.

In some embodiments, the screening step comprises transforming a phage library into cloning bacterial strain, such as an E. coli strain, collecting the transformants, growing to saturation, and characterizing barcoded junctions derived from the phage library.

In some embodiments, the DNA fragments, or at least about 50%, 60%, 70%, 70%, 80%, or 90% DNA fragments, have an average size of from about 1.0 kilobasepairs (kbp), 1.5 kbp, 2.0 kbp, 2.5 kbp, 3.0 kbp, 3.5 kbp, 4.0 kbp, 4.5 kbp, 5.0 kbp, 5.5 kbp, or 6.0 kbp, or an average size within the range of any two preceding values. In some embodiments, the DNA fragments, or at least about 50%, 60%, 70%, 70%, 80%, or 90% DNA fragments, have sizes that fall within a range of any two of the following values: about 1.0 kbp, 1.5 kbp, 2.0 kbp, 2.5 kbp, 3.0 kbp, 3.5 kbp, 4.0 kbp, 4.5 kbp, 5.0 kbp, 5.5 kbp, and 6.0 kbp. In some embodiments, the vector is a medium copy vector.

In some embodiments, the providing one or more DNA barcoded overexpression strain libraries using DNA of the host organism and/or phage comprises shearing genomes of one or more bacteriophages inserting a barcoded transposon into a host organism, such as using the method taught in Example 1, wherein the bacteriophages(s) can be any bacteriophages(s) which correspond to a single host, such as any described in Table 1.

In some embodiments, there is one species of host organism and a plurality of bacteriophage species wherein each bacteriophage species is capable of infecting the host organism. In other embodiments, there are a plurality of host organism species and one bacteriophage species wherein the bacteriophage species is capable of infecting each host organism species in the plurality of host organism species.

In some embodiments, the functions comprise one or more of the following: recognition, entry, replication, and host lysis.

Both technologies employ a high-throughput DNA barcode sequencing readout (BarSeq) that enable cost effective and genome-wide assays of gene fitness in a single-pot assay.

In some embodiments, each barcode is a barcode taught in U.S. Patent Applications Pub. No. 2018/0030435, hereby incorporated by reference in its entirety.

In some embodiments, the providing and/or screening steps are automated and/or high throughout. In some embodiments, each individual host organism and/or phage sample is provided and/or screened in a format configured for automated and/or high throughout processing and/or handling, such as a 96-well format.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Workflow for screening receptors for phages, phage-tail like particles, peptides, bacteriocins, antibiotics, metals and predatory bacteria.

FIG. 2. Screening for phage resistance via genome-wide LOF libraries. Different dilutions of phages (multiplicity of infection) and high scoring genes are shown. This is a snapshot of the genome-wide data. Gene score panel is shown on the top of the heatmap.

FIG. 3. Screening for phage resistance via genome-wide GOF Dub-seq library. Different dilutions of phages (multiplicity of infection) and high scoring genes are shown. This is a snapshot of the genome-wide data. Gene score panel is shown on the top of the heatmap.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “barcode” or “barcodes” can refer to nucleic acid codes or sequences associated with a target within a sample. A barcode can be, for example, a nucleic acid label. A barcode can be an entirely or partially amplifiable barcode. A barcode can be entirely or partially sequenceable barcode. A barcode can be a portion of a native nucleic acid that is identifiable as distinct. A barcode can be a known sequence. A barcode can be a random sequence. A barcode can comprise a junction of nucleic acid sequences, for example a junction of a native and non-native sequence. As used herein, the term “barcode” can be used interchangeably with the terms, “index”, “tag,” or “label-tag.” Barcodes can convey information. For example, in various embodiments, barcodes can be used to determine an identity of a nucleic acid, a source of a nucleic acid, an identity of a cell, and/or a target.

As used herein, a “nucleic acid” can generally refer to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g. altered backgone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone of the nucleic acid can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,′:4,5)pyrrolo[2,3-d]pyrimidin-2-one).

Methods of Quantitative Analysis of Nucleic Acid Target Molecules

Some embodiments disclosed herein provide methods of constructing an expression library from a plurality of nucleic acid fragments. In some embodiments, the plurality of nucleic acid fragments are from a single cell, a plurality of cells, a tissue sample, a virus, a fungus, or any combination thereof. The nucleic acid fragments can be DNA, such as genomic DNA, cDNA, and the likes; or RNA, such as mRNA, microRNA, tRNA, rRNA, and the likes. In some embodiments, the plurality of nucleic acid fragments can be a plurality of genomic fragments. In some embodiments, the plurality of genomic fragments can comprise a completely or partially sequenced genome, a single cell genome, a viral genome, a bacterial genome, a metagenome, or any combination thereof. In some embodiments, the plurality of nucleic acid fragments are from a single cell, a plurality of cells, a tissue sample, a virus, a fungus, or any combination thereof. The nucleic acid fragments can have a variety of sizes. For example, the plurality of nucleic acid fragments can have an average size that is, is about, is less than, is greater than, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, or a range between any two of the above values. In some embodiments, the nucleic acid fragments can be obtained by a fragmenting treatment, including but not limited to enzymatic treatment such as restriction enzyme digestion, physical treatment such as sonication, etc.

In some embodiments, the methods comprise providing a plurality of vectors. In some embodiments, each vector comprises one or more barcodes. The plurality of vectors can comprise at least about 100, 1,000, 10,000, 100,000, 1,000,000, or more vectors. In some embodiments, each vector comprises two barcodes. The barcode, or the two barcodes, can be selected from a set of unique barcodes. The barcode or the two barcodes can be completely random in sequence which can be sequenced before (or after) nucleic acid fragment cloning. In some embodiments, the plurality of vectors can be characterized so that each vector is identified with a unique barcode or a unique combination of two or more barcodes. In some embodiments, the characterization of the vectors comprises sequencing at least a portion of the one or more barcodes. In some embodiments, the two barcodes in a vector are next to each other. In some embodiments, the two barcodes are separated by one or more restriction sites. In some embodiments, the two barcodes are separated by one or more selection marker genes.

A barcode can comprise a nucleic acid sequence that provides identifying information for the specific nucleic acid fragment associated with the barcode. A barcode can be at least about 1, 2, 3, 4, 5, 10, 15, 20, 25, 30, 35, 40, 45, 50, or more nucleotides in length. A barcode can be at most about 300, 200, 100, 90, 80, 70, 60, 50, 40, 30, 20, 15, 12, 10, 9, 8, 7, 6, 5, 4, or fewer nucleotides in length. In some embodiments, there may be as many as 10⁶ or more different barcodes in the set of unique barcodes. In some embodiments, there may be as many as 10⁵ or more different barcodes in the set of unique barcodes. In some embodiments, there can be as many as 10⁴ or more different barcodes in the set of unique barcodes. In some embodiments, there can be as many as 10³ or more different barcodes in the set of unique barcodes. In some embodiments, there can be as many as 10² or more different barcodes in the set of unique barcodes.

In some embodiments, a barcode can be flanked by a pair of binding sites for two universal primers. The two universal primers can be the same or different. In some embodiments, each barcode of the plurality of vectors is flanked by the same pair of binding sites.

An expression vector includes vectors capable of expressing DNA's that are operatively linked with regulatory sequences, such as promoter regions, that are capable of effecting expression of such DNA fragments. Thus, an expression vector refers to a recombinant DNA or RNA construct, such as a plasmid, a phage, a virus, a recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome. The vector can be a variety of suitable replication units, including but not limited to: plasmids, viral vectors, cosmids, fosmids, and artificial chromosomes. In some embodiments, the vector is a broad-host-range replication vector. For example, there are a wide range of broad-host plasmids, cosmids and fosmids available based on IncQ, IncW, IncP, and pBBR1-based systems that can replicate in diverse microbes (Lale et al., (2011) Broad-host-range plasmid vectors for gene expression in bacteria. Strain engineering: Methods and protocols (Ed., James Williams), Methods in molecular biology, Vol 756, Chapter 19, 327-343).

In some embodiments, the vector can comprise a promoter sequence, such as a constitutive promoter, a synthetic promoter, an inducible promoter, an endogenous promoter, an exogenous promoter, or any combination thereof. In some embodiments, the vector can comprise a poly-A sequence. In some embodiments, the vector can comprise a translation termination sequence, and/or a transcription termination sequence. In some embodiments, the vector can further encode a tag sequence.

In some embodiments, the methods comprise inserting the plurality of nucleic acid fragments into the plurality of vectors to generate a plurality of expression vectors. In some embodiments, the plurality of nucleic acid fragments can be ligated with one or more adaptors before inserting into the vectors. In some embodiments, the one or more adaptors comprise one or more barcodes and/or one or more binding sites for a universal primer. A barcode alone, or two barcodes in combination, can be associated with the nucleic acid fragment that is inserted into the vector. For example, the nucleic acid fragment inserted into the vector can be flanked by the two barcodes.

Inserting the nucleic acid fragments can comprise ligation, such as blunt end ligation. In some embodiments, the vectors can be digested with a restriction enzyme to linearize the vectors. In some embodiments, the linearized vectors are blunt-ended before the ligation with the nucleic acid fragments.

In some embodiments, the methods comprise transforming the plurality of expression vectors into a host organism. A host organism is a bacterial cell. In some embodiments, the methods comprise growing the transformed host organism under a selection condition, so that only the host organisms transformed with the expression vector can survive. In some embodiments, the bacterial cells are or comprise Gram-negative cells, and in some embodiments, the bacterial cells are or comprise Gram-positive cells. Examples of bacterial cells of the invention include, without limitation, Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus Enterococcus faecalis, Bacillus coagulans, Bacillus ceretus, Bacillus popillae, Synechocystis strain PCC6803, Bacillus liquefaciens, Pyrococcus abyssiSelenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis.

In some embodiments, the host organism is one or more hosts described in Table 1 herein, and the bacteriophage is one or more bacteriophages described in Table 1 which correspond to the host.

With rapid rise in instances of antibiotic resistant bacteria and other deleterious effects caused by antibiotics on commensal healthy microbiome, there is an increased awareness to find novel solutions to antibiotics. One proposed alternative is to use bacterial viruses or bacteriophages that prey and kill pathogenic bacteria. However, decades of research has shown that bacteria use a spectrum of strategies to protect themselves from phage infection. These interaction studies between bacteria and phages have been largely performed on few key model bacterium/phage strains. Even in well studied model systems, we still do not know the full breadth of host resistance mechanisms to diverse phages. To realize the widespread successful practice of phage therapy, we need to know the phage resistance mechanisms and understand factors important in host infection pathways. Unfortunately, the current methods used to detect phage receptors suffer from tedious sample preparations, expensive sequencing methods and low throughout assays. We need new technologies that are quantitative, scalable, economical, can be applied to diverse hosts and phages at different multiplicity of infection. Such genome-wide approaches for identifying these phage-host interaction determinants would be highly valuable for obtaining systems-level understanding of phage infection pathways and phage-resistance phenotypes ands such approaches are necessary to develop phage-based strategies for precise microbial community engineering. In addition, by knowing phage receptors, it would be possible in the future to make rationally designed cocktails of phages that target different host pathways and eliminate the possibility of phage resistance.

Recently, we have developed two genetic technologies that enable fast and effective genome-wide screens for gene function, and are suitable for discovering host genes crucial in phage infection. The first, randomly barcoded transposon sequencing (RB-TnSeq,) method, generates strain libraries for screening loss-of-function mutant phenotypes. The second method generates DNA barcoded overexpression strain libraries (Dub-seq) method using DNA of the host or phage and permits gain-of-function assays. Both technologies employ a high-throughput DNA barcode sequencing readout (BarSeq) that enable cost effective and genome-wide assays of gene fitness in a single-pot assay. These method decouple the genetic characterization from phenotype determination steps, and enable the entire pipeline of characterization cheaper, quantitative, less laborious and scalable than any currently available technologies. This disclosure details on invention of doing high throughput screens to discover phage receptors and other host factors that are important in phage infection and resistance. These competitive fitness assays can also be used for screening and discovering resistance factors for phage-like bacteriocins, bacterial predators, antimicrobial peptides and enzymes.

This disclosure details on invention of doing high throughput screens to discovery host factors important in phage infection or bacterial lysis by phage like particles including peptide bacteriocins and antimicrobial enzymes. Herein are described two technologies.

Bacteria use a spectrum of strategies to protect themselves from phage infection. The mechanisms of these phage-host interaction strategies have been largely derived from focused studies on a handful of individual bacterium/phage systems. It has been realized that genome-wide approaches for identifying these phage-host interaction determinants would be highly valuable for obtaining systems-level understanding of phage infection pathways and phage-resistance phenotypes and we are in need of methods that are easily transferable to new systems. Such approaches are necessary to develop phage-based strategies for precise microbial community engineering. Indeed, a number of studies have highlighted the importance of high-throughput technologies applied to phage engineering, genome assembly and significance of uncovering host-specificity determinants for further phage engineering applications.

We have developed two genetic technologies that enable fast and effective genome-wide screens for gene function, and are suitable for discovering host genes crucial in phage infection. The first, randomly barcoded transposon sequencing (RB-TnSeq) method, generates strain libraries for screening loss-of-function mutant phenotypes. The second method generates DNA barcoded overexpression strain libraries (Dub-seq) method using DNA of the host or phage and permits gain-of-function assays. Both technologies employ a high-throughput DNA barcode sequencing readout (BarSeq) that enable cost effective and genome-wide assays of gene fitness in a single-pot assay.

These method decouple the genetic characterization from phenotype determination steps, and enable the entire pipeline of characterization cheaper, quantitative, less laborious and scalable than any currently available technologies. For these two loss-of-function and gain-of-function screens to work, we had to optimize the multiplicity of infection, time of assay, sample preparation and data analysis pipelines.

Drug companies (Genentech, Roche, Dupont, J & J, Novartis etc) and phage therapy (C3J, Enbiotix, Locus, BiomX, Eligo.Pylum Biosciences, Omnilytic, AmpliPhi) companies are more likely use the technology.

Our combination of loss-of-function and gain of function methods enable researchers to gain mechanistic insights into antimicrobial compounds, phages, and phage like particles. This enables in designing rational cocktail formulation. Currently this is done in a very ad hoc fashion and subjected to lot of failures.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### High-Throughput Genome-Wide Screen to Discover Host and Phage Factors Important in Phage Infection and Resistance Elucidates Rational Method to Formulate Phage Cocktails

Bacteria use a spectrum of strategies to protect themselves from phage infection. The mechanistic insights into these phage-host interaction strategies have been largely derived from focused studies on a handful of individual bacterium/phage systems and low-throughout approaches. It has been realized that genome-wide approaches for identifying these phage-host interaction determinants would be highly valuable for obtaining systems-level understanding of full breadth of resistance mechanisms available to bacteria, and identify the degree of specificity for each bacterial resistance mechanism across diverse phage types. Such approaches may then enable rational phage cocktail formulation for therapeutic applications and microbial community manipulation. Here, we apply recently developed genome-wide loss-of-function and gain-of-function genetic technologies to canonical, phylogenetically diverse double-stranded DNA phages infecting E. coli strains K-12. We discover a core set of host genes that are conditionally essential for phage infection and play an important role in phage resistance. We uncover the commonality and distinctiveness in these genetic determinants across different phages. We also extend the gain-of-function genetic technology to overexpress fragments of phage genomes and develop a method for systematic study of superinfection mechanism, where in one phage selectively inhibits infection by another phage.

Overall, this study provides a systematic workflow for developing next generation phage characterization platform for studying phage biology. This characterization platform also enables rational formulation of phage cocktails important in phage therapeutic applications and acts as a hypothesis generator in phage engineering applications. By gaining insights into phage superinfection exclusion mechanisms scientists can design better phage cocktails, which can be synergistic in overcoming target pathogen and also understand failed phage treatments. The characterization pipeline can be easily extended to study host factors important in phage-tail like bacteriocins, peptides, antibiotics, metals and bacterial predators.

We published two genetic technologies that enable fast and effective genome-wide screens for gene function, and are suitable for discovering host genes or receptors crucial in phage infection. The first, randomly barcoded transposon sequencing (Wetmore, et al., MBio, 6, 3, e00306-15, 2015; hereby incorporated by reference in its entirety), generates strain libraries for screening loss-of-function mutant phenotypes in nonessential genes. The second method generates DNA barcoded overexpression strain libraries, such as Dual barcoded Shotgun Expression library sequencing (Dub-seq), using genome fragments of the host and permits gain-of-function assays in pooled competitive fashion (Mutalik et al., Nat Communications, 10, 308, 2019; hereby incorporated by reference in its entirety). Both technologies employ the same high-throughput DNA barcode sequencing readout (Barseq) that enables cost effective, less-laborious, quantitative genome-wide assays of gene fitness in a single-pot across diverse conditions. As an example of efficiency, we have been able to apply RB-TnSeq across 32 diverse bacteria in over 4800 genome-wide condition assays to make 18.7 million gene phenotype measurements in just over a couple of years (Price et al., Nature, 557, 503-509, 2018; hereby incorporated by reference in its entirety). Similarly, for gain-of-function Dub-seq technology, we performed 155 genome-wide fitness assays in 52 experimental conditions including antibiotics and metals, and identified overexpression phenotypes for 813 E. coli genes (Mutalik et al., Nat Communications, 10, 308, 2019).

These technologies can also be useful for studying superinfection mechanism, in which preexisting phage infection prevents a secondary infection by the same or different phage. Even though it has been hypothesized that this mechanism is widespread in diverse viruses, only few of superinfection exclusion systems are known to date (Lu and Henning, Trends Microbiol 2, 137-139, 1994; Barrangou and van der Oost, EMBO J 34, 134-135, 2015; Bondy-Denomy, J. et al. ISME J 10, 2854-2866, 2016; hereby incorporated by reference in their entireties). It appears that these genes or systems are encoded either on prophages or lytic phage genomes themselves, but how widespread these superinfection mechanisms in lytic phages and how they impact host fitness is less understood. Two well-studied examples for lytic bacteriophage are: E. coli phage T4 encodes two systems (Imm and Sp), which inhibit DNA injection of T4 and other T-even-like phages (Lu and Henning, Trends Microbiol 2, 137-139, 1994; Lu and Henning, J Virol 63, 3472-3478, 1989; hereby incorporated by reference in their entireties). T5 codes for Llp protein that is formed in preinfected cells and blocks its own receptor, thereby preventing superinfection by other T5 phages (Decker et al., Mol Microbiol 12, 321-332, 1994; hereby incorporated by reference in its entirety).

Here we have employed these two technologies (RB-TnSeq, Dub-seq) as a demonstration of a “portable” and “scalable” technology for probing host/phage interactions mechanisms in bacteria. As a demonstration of this approach, we have used E. coli strain K-12 and 6 diverse canonical double-stranded DNA phages. By comparing results of experiments across phage-host combinations we uncovered conserved genetic determinants of phage specificity, resistance and propagation, as well as those that differentiate among bacteria and phage strains. We show that our data is consistent with known biology, thus validating the results, but also are able to yield novel phage-resistance mechanisms. This study provides a foundation for developing rationally designed phage cocktail for therapeutic applications. Superinfection study also provided us with different phage genes that inhibit infection by other phages. By extending these studies to other pathogen bacteria-phage combinations along with other antibacterial biological agents/chemicals such as phage-tail like bacteriocins, peptides, antibiotics, metals and bacterial predators, we would be able to create a knowledge base, that enables us to create rational combination of antibacterial cocktails powered by machine learning algorithms for treating antibiotic resistant pathogens. Methods Phages:

We sourced diverse E. coli phages belong to diverse classes, each having overlapping but distinct mechanisms of recognition, entry, replication and host lysis. These included T-phages (T2, T3, T4, T5, T6, T7 phages) and used in independent fitness screens at different multiplicity of infection for each phage-host combination. Most of these phages have been widely studied and reviewed (Table 1, Silva et al., FEMS Microbiology letters, 363, 2016, fnw002; Letarov and Kulikov, Biochemistry (Moscow), 82, 13, 1632-1658, 2017; hereby incorporated by reference in their entireties). Among phages we used in this study, genome-wide screens have been reported earlier on T4 and T7 (Qimron et al., PNAS, 103, 50, 19039-19044, 2006; Rousett, et al., PLoS Genet 14, 11, e1007749, 2018; hereby incorporated by reference in their entireties) providing an avenue for comparison with our screens.

TABLE 1
Recent reviews highlights discovery of phage receptors for few model hosts
over the period of decades (Silva et al., FEMS Microbiology letters, 363,
2016, fnw002; Letarov and Kulikov, Biochemistry (Moscow), 82, 13,
1632-1658, 2017; hereby incorporated by reference in their entireties)

| Phages | Family | Main host | Receptor(s) |
|---|---|---|---|
| γ | Siphoviridae | Bacillus anthracis | Membrane surface-anchored |
| | | | protein gamma phage receptor |
| | | | (GamR) |
| SPP1 | Siphoviridae | Bacillus subtilis | Glucosyl residues of |
| | | | poly(glycerophosphate) |
| | | | on WTA for reversible binding |
| | | | and membrane protein YueB |
| | | | for irreversible binding |
| φ29 | Podoviridae | Bacillus subtillus | Cell WTA (primary receptor) |
| Bam35 | Tectiviridae | Bacillus | N-acetyl-muramic acid |
| | | thuringiensis | (MumNAc) of peptidoglycan |
| | | | in the cell wall |
| LL-H | Siphoviridae | Lactobacillus | Glucose moiety of LTA for |
| | | delbruechii | reversible adsorption and |
| | | | negatively charged glycerol |
| | | | phosphate group of the |
| | | | LTA for irreversible binding |
| B1 | Siphoviridae | Lactobacillus | Galactose component |
| | | plantarum | of the wall polysaccharide |
| B2 | Siphoviridae | Lactobacillus | Glucose substituents in |
| | | plantarum | teichoic acid |
| S | Siphoviridae | Lactococcus | Rhammosa^{a} moieties in the |
| 13 | | lactis | cell wall peptidoglycan for |
| c2 | | | reversible binding and |
| h | | | membrane phage infection |
| ml3 | | | protein (PIP) for |
| kh | | | irreversible binding |
| L | | | |
| φLC3 | Siphoviridae | Lactococcus | Cell wall polysaccharides |
| TP901term | | lactis | |
| TP901-1 | | | |
| p2 | Siphoviridae | Lactococcus | Cell wall saccharides for |
| | | lactis | reversible attachment and |
| | | | pellicle^{b} phosphohexa- |
| | | | saccharide motifs for |
| | | | irreversible adsorption |
| A511 | Myouiridae | Listeria | Peptidoglycan (murein) |
| | | monocytogenes | |
| A118 | Siphoviridae | Listeria | Glucosaminyl and |
| | | monocytogenes | rhamnosyl components |
| | | | of ribitol teichoic acid |
| A500 | Siphoviridae | Listeria | Glucosaminyl residues |
| | | monocytogenes | in teichoic acid |
| φ812 | Myoviridae | Staphyloccus | Anionic backbone of WTA |
| φK | | aureus | |
| 52A | Siphoviridae | Staphyloccus | O-acetyl group from the 6- |
| | | aureus | position of muramic acid |
| | | | residues in murein |
| W | Siphoviridae | Staphyloccus | N-acetylglucosamine |
| φ13 | | aureus | (GlcNAc) glycoepitope |
| φ47 | | | on WTA |
| φ77 | | | |
| φSa2m | | | |
| φSLT | Siphoviridae | Staphyloccus | Poly(glycerophosphate) |
| | | aureus | moiety of LTA |

(a) Receptors that bind to RBP of phages

| | | | |
|---|---|---|---|
| φCr30 | Myoviridae | Caulobacter | Paracrystalline surface (S) |
| | | crescentus | layer protein |
| 434 | Siphoviridae | Escherichia | Protein Ib (OmpC) |
| | | coli | |
| BF23 | Siphoviridae | Escherichia | Protein BtuB (vitamin B₁₂ |
| | | coli | receptor) |
| K3 | Myoviridae | Escherichia | Protein d or 3A |
| | | coli | (OmpA) with LPS |
| K10 | Siphoviridae | Escherichia | Outer membrane protein |
| | | coli | LamB (maltodextran |
| | | | selective channel) |
| Me1 | Myoviridae | Escherichia | Protein r (OmpC) |
| | | coli | |
| Mu G(+) | Myoviridae | Escherichia | Terminal Glcα-2Glcα1-or |
| | | coli | GlcNAcα1-2Glcα1-of the LPS |
| Mu G(−) | Myoviridae | Escherichia | Terminal glucose with a β1,3 |
| | | coli | glycosidic linkage |
| | | Erwinia | Terminal glucose linked |
| | | | in β1,6 configuration |
| M1 | Myoviridae | Escherichia | Protein OmpA |
| | | coli | |
| Ox2 | Myoviridae | Escherichia | Protein OmpA^{a} |
| | | coli | |
| ST-1 | Microviridae | Escherichia | Terminal Glcα-2Glcα1-or |
| | | coli | GlcNAcα1-2Glcα1-of |
| | | | the LPS |
| TLS | Siphaviridae | Escherichia | Antibiotic efflux protein |
| | | coli | TolC and the inner |
| | | | core of LPS |
| TuIa | Myoviridae | Escherichia | Protein 1a (OmpF) |
| | | coli | with LPS |
| TuIb | Myoviridae | Escherichia | Protein 1b (OmpC) |
| | | coli | with LPS |
| TuII^{a} | Myoviridae | Escherichia | Protein II^{a} (OmpA) |
| | | coli | with LPS |
| T1 | Siphoviridae | Escherichia | Proteins TonA (FhuA, |
| | | coli | involved in ferri- |
| | | | chrome uptake) and TonB^{b} |
| T2 | Myoviridae | Escherichia | Protein Ia (OmpF) with |
| | | coli | LPS and the outer membrane |
| | | | FadL (involved in the uptake |
| | | | of long-chain fatty acids) |
| T3 | Podoviridae | Escherichia | Glucosyl-α-1,3-glucose |
| | | coli | terminus of rough LPS |
| T4 | Myoviridae | Escherichia | Protein O-8 (OmpC) |
| | | coli K-12 | with LPS |
| | | Escherichia | Glucosyl-α-1,3 glucose |
| | | coli B | terminus of rough LPS |
| T5 | Siphoviridae | Escherichia | Polymannose sequence in |
| | | coli | the O-antigen and |
| | | | protein FhuA |
| T6 | Myoviridae | Escherichia | Outer membrane protein Tsx |
| | | coli | (involved in nucleo- |
| | | | side uptake) |
| T7 | Podoviridae | Escherichia | LPS^{a} |
| | | coli | |
| U3 | Microviridae | Escherichia | Terminal galactose |
| | | coli | residue in LPS |
| λ | Siphoviridae | Escherichia | Protein LamB |
| | | coli | |
| φX174 | Microviridae | Escherichia | Terminal galactose in the |
| | | coli | core oligosaccharide of |
| | | | rough LPS |
| φ80 | Siphoviridae | Escherichia | Proteins FhuA and TonB^{b} |
| | | coli | |

(a) Receptors that bind to RBP of phages

| | | | |
|---|---|---|---|
| PM2 | Corticoviridae | Pseudo- | Sugar moieties on the |
| | | alteromonas | cell surface^{d} |
| E79 | Myoviridae | Pseudomonas | Core |
| | | aeruginosa | polysaccharide of LPS |
| JG004 | Myoviridae | Pseudomonas | LPS |
| | | aeruginosa | |
| φCTX | Myoviridae | Pseudomonas | Core polysaccharide of |
| | | aeruginosa | LPS, with emphasis on |
| | | | L-rhamnose and D- |
| | | | glucose residues in the |
| | | | outer core |
| φPLS27 | Podoviridae | Pseudomonas | Galactosamine- |
| | | aeruginosa | alanine region of the |
| | | | LPS core |
| φ13 | Cystoviridae | Pseudomonas | Truncated O-chain |
| | | syringae | of LPS |
| ES18 | Siphoviridae | Salmonella | Protein FhuA |
| Gifsy-1 | Siphoviridae | Salmonella | Protein OmpC |
| Gifsy-2 | | | |
| SPC35 | Siphoviridae | Salmonella | BtuB as the main receptor and |
| | | | O12-antigen as adsorption- |
| | | | assisting apparatus |
| SPN1S | Podoviridae | Salmonella | O-antigen of LPS |
| SPN2TCW | | | |
| SPN4B | | | |
| SPN6TCW | | | |
| SPN8TCW | | | |
| SPN9TCW | | | |
| SPN13U | | | |
| SPN7C | Siphoviridae | Salmonella | Protein BtuB |
| SPN9C | | | |
| SPN10H | | | |
| SPN12C | | | |
| SPN14 | | | |
| SPN17T | | | |
| SPN18 | | | |
| vB_SenM- | Myoviridae | Salmonella | Protein OmpC |
| S16 (S16) | | | |
| L-413C | Myoviridae | Yersinia pestia | Terminal GlcNAc residue of |
| P2 vir1 | | | the LPS outer core. HepII/HepIII |
| | | | and Hep1/Glc residues are |
| | | | also involved in receptor |
| | | | activity^{e} |
| φJA1 | Myoviridae | Yersinia pestia | Kdo/Ko pairs of inner core |
| | | | residues. LPS outer and inner |
| | | | core sugars are also involved |
| | | | in receptor activity^{e} |
| T7_{Yp} | Podoviridae | Yersinia pestia | Hep1/Glc pairs of inner core |
| Y (YpP-Y) | | | residues. HepII/HepIII and Kdo/Ko |
| | | | pairs are also involved in |
| | | | receptor activity^{e} |
| Pokrovskaya | Podoviridae | Yersinia pestia | HepII/HepIII pairs of inner |
| YepE2 | | | core residues. HepI/Glc |
| YpP-G | | | residues are also involved |
| | | | in receptor activity^{e} |
| φA1122 | Podoviridae | Yersinia pestia | Kdo/Ko pairs of inner core |
| | | | residues. HepI/Glc residues |
| | | | are also involved in |
| | | | receptor activity^{e} |
| PST | Myoviridae | Yersinia | HepII/HepIII pairs of |
| | | pseudo- | inner core residues^{a} |
| | | tuberculosis | |

(b) Receptors in the O-chain structure that are enzymatically cleaved by phages

| | | | |
|---|---|---|---|
| Ω8 | Podoviridae | Escherichia | The α-1,3-mannosyl linkages |
| | | coli | between the trisaccharide |
| | | | repeating unit α-mannosyl- |
| | | | 1,2-α-mannosyl-1,2-mannose |
| c341 | Podoviridae | Salmonella | The O-acetyl group in the |
| | | | mannosyl-rhamnosyl- |
| | | | O-acetylgalactose |
| | | | repeating sequence |
| P22 | Podoviridae | Salmonella | α-Rhmanosyl 1-3 galactose |
| | | | linkage of the O-chain |
| e³⁴ | Podoviridae | Salmonella | [-β-Gal-Man-Rha-] |
| | | | polysaccharide units of |
| | | | the O-antigen |
| Sf6 | Podoviridae | Shigella | Rha II 1-α-3 Rha III |
| | | | linkage of the |
| | | | O-polysaccharide. |

(a) Receptors in flagella

| | | | |
|---|---|---|---|
| SPN2T | Siphoviridae | Salmonella | Flagella protein FliC |
| SPN3C | | | |
| SPN8T | | | |
| SPN9T | | | |
| SPN11T | | | |
| SPN13B | | | |
| SPN16C | | | |
| SPN4S | Siphoviridae | Salmonella | Flagellin proteins FliC or FljB |
| SPN5T | | | |
| SPN6T | | | |
| SPN19 | | | |
| iEPS5 | Siphoviridae | Salmonella | Flagellal molecular ruler |
| | | | protein FliK |

(b) Receptors in pili and mating pair formation structures

| | | | |
|---|---|---|---|
| φChK | Siphoviridae | Caulobacter | Initial contact between phage |
| φCh13 | | crescentus | head filament and host's flagellum |
| | | | followed by pili portals on the |
| | | | cell pole. |
| Fd | Inoviridae | Escherichia coli | Tip of the F pilus followed |
| Pf | | | by TolQRA complex in |
| f1 | | | membrane after pilus |
| M13 | | | retraction |
| PRD1 | Tectiviridae | Escherichia coli | Mating pair formation |
| | | | (Mpf) complex in the |
| | | | membrane |
| φ6 | Cystoviridae | Psuedomonas | Sides of the type IV pilus |
| MPK7 | Podoviridae | Pseudomonas | Type IV pili (TFP) |
| | | aeruginosa | |
| MP22 | Siphoviridae | Pseudomonas | Type IV pili (TFP) |
| | | aeruginosa | |
| DMS3 | Siphoviridae | Pseudomonas | Type IV pili (TFP) |
| | | aeruginosa | |

(c) Receptors in bacterial capsules

| φChK | Siphoviridae | Caulobacter | | Initial contact between phage head | |
|---|---|---|---|---|---|
| φCh13 | | crescentus | | filament and host's flagellum | |
| | | | | followed by pili portalis on the | |
| | | | | cell pole | |
| Fd | Inoviridae | Escherichia coli | | Tip of the F pilus followed | |
| Pf | | | | by TolQRA complex in | |
| f1 | | | | membrane after pilus | |
| M13 | | | | retraction | |
| PRD1 | Tectiviridae | Escherichia coli | | Mating pair formation | |
| | | | | (Mpf) complex in the | |
| | | | | membrane | |
| φ6 | Cystoviridae | Psuedomonas | | Sides of the type IV pilus | |
| MPK7 | Podoviridae | Pseudomonas | | Type IV pili (TFP) | |
| | | aeruginosa | | | |
| MP22 | Siphoviridae | Pseudomonas | | Type IV pili (TFP) | |
| | | aeruginosa | | | |
| DMS3 | Siphoviridae | Pseudomonas | | Type IV pili (TFP) | |
| | | aeruginosa | | | |
| 29 | Podoviridae | Escherichia coli | | Endoglycosidase hydrolysis | |
| | | | | in β-D-glucosido-(1-3)-D- | |
| | | | | glucoronic acid bonds in | |
| | | | | the capsule composed of | |
| | | | | hexasaccharides repeating | |
| | | | | units | |
| K11 | Podoviridae | Klebsiella | | Hydrolysis of | |
| | | | | β-D-glucosyl-(1-3)-β-D- | |
| | | | | glucuronic acid linkages. | |
| | | | | The phage is also able to | |
| | | | | cleave α-D-galactosyl- | |
| | | | | (1-3)-β-D-glucose bonds | |
| Vl I | Myoviridae | Salmonella | | Acetyl groups of the Vl | |
| | | | | exopolysaccharide capsule | |
| | | | | (a polymer of α-1,4-linked | |
| | | | | N-acetyl galactosaminuronate) | |
| Vl II | Siphoviridae | Salmonella | | Acetyl groups of the Vl | |
| | | | | exopolysaccharide capsule | |
| | | | | (a polymer of α-1,4-linked | |
| | | | | N-acetyl galactosaminuronate) | |
| Vl III | Podoviridae | Salmonella | | Acetyl groups of the Vl | |
| Vl IV | | | | exopolysaccharide capsule | |
| Vl V | | | | (a polymer of α-1,4-linked | |
| Vl VI | | | | N-acetyl galactosaminuronate) | |
| Vl VII | | | | | |
| Bacterio- | | Genus/ | | Primary | Secondary |
| phage | Family | group | Host | receptor | receptor |
| T1 | S | T1-like | E. coli | ? | FhuA (requires |
| | | | | | TonB) |
| T4 | M | T4-like | E. coli, | OmpC | LPS core |
| | | | Shigella | | |
| T5 | S | TS-like | E. coli | LPS | FhuA |
| | | | | O-antigen | |
| | | | | (polyman- | |
| | | | | nose)- | |
| | | | | optionally | |
| BF23 | S | TS-like | E. coli | LPS? | BtuB |
| λ | S | lamb- | E. coli | OmpC | LamB |
| | | doids | | | |
| | | λ-like) | | | |
| P22 | P | lamb- | E. coli | LPS | LPS? |
| | | doids | | O-antigen | |
| | | (P22- | | | |
| | | like) | | | |
| Sf6 | P | ? | Shigella | LPS | OmpA, |
| | | | Flexneri | | OmpC |
| N4 | P | N4-like | E. coli | ? | NfrA |
| G7C | P | N4-like | E. coli | LPS | unknown |
| | | | 4s | O-antigen | (OmpA and |
| | | | | O22-like | ?) |
| Alt63 | P | N4-like | E. coli | LPS | unknown |
| | | | 4s | O-antigen | (OmpA and |
| | | | | | ?) |
| CP81 and | M | ? | Campylo- | exopoly- | ? |
| related | | | bacter | saccharide; | |
| phages | | | jejuni | modification | |
| | | | NCTC12658 | of the MeOPN | |
| | | | | type is important | |
| | | | | for some phages | |
| CP220 and | M | ? | Campylo- | motile | ? |
| related | | | bacter | flagellum | |
| phages | | | jejuni | | |
| | | | NCTC12658 | | |
| NCTC12673 | | | Campylo- | glycosylated | ? |
| | | | bacter | flagellin | |
| | | | jejuni | | |
| VP5 | ? | ? | Vibria | ? | OmpW |
| | | | cholerae | | |
| | | | O1 El Tor | | |
| phiR1-37 | ? | ? | Yersinia | LPS O-antigen | ? |
| | | | similis O9 | | |
| | | | and other | | |
| | | | Yersinia | | |
| SSU5 | S | | Salmonella | LPS external | ? |
| | | | enterica, | core | |
| | | | Shigella, | | |
| | | | E. coli | | |
| | | | K-12 | | |
| S16 | M | T4-like | Salmonella | OmpC | ? |
| VP4 | | | Vibrio | LPS O-antigen | |
| | | | cholerae | | |
| | | | O1 El Tor | | |
| phiX216 | M | P2-like | Burkholderia | LPS O-antigen | ? |
| | | | mallei, | of B. mallei | |
| | | | B. pseudomallei | | |
| SPC35 | S | T5-like | Salmonella | LPS O-antigen | BtuB |
| | | | enterica | | |
| | | | serovar | | |
| | | | Typhimurium | | |
| SPN10H | S | T5-like | S. enterica | LPS? | BtuB |
| (and 6 | | | serovar | | |
| other | | | Typhimurium | | |
| isolates) | | | | | |
| SPN2T | S | ? | S. enterica | flagellum | ? |
| | | | serovar | | |
| | | | Typhimurium | | |
| SPN1S | P | ? | S. enterica | LPS | ? |
| (and 6 | | | serovar | | |
| other | | | Typhimurium | | |
| isolates) | | | | | |
| phiA1122 | P | T7-like | Yersinia | ? | Hep/Glc- |
| | | | pestis, | | Kdo/Ko |
| | | | Y. pseudo- | | regions of |
| | | | tuberculosis | | LPS core |
| phiCb13 and | S | ? | Caulobacter | flagellum | pili portal |
| phiCbK | | | crescentus | | |
| Mlo1 | S | ? | Mesorhizobium | LPS | LPS (?) |
| | | | loti | | |
| ST27, ST29, | ? | un- | S. enterica | ? | TolC |
| ST35 (and | | known | serovar | | |
| probably 14 | | | Typhimurium | | |
| more un- | | | | | |
| characterized | | | | | |
| phages) | | | | | |
| IMM-01 | S | ? | enterotoxi- | ? | CS7 |
| | | | genic E. coli | | colonization |
| | | | (ETEC) | | factor (pilus) |
| VP3 | P | T7-like | V. cholerae | LPS core | |
| | | | O1 El Tor | | |
| EPS7 | S | TS-like | S. enterica, | ? | BtuB |
| | | | E. coli | | |
| 37 isolates | ? | lamb- | E. coli (?) | ? | FhuA |
| lambdoid | | doids | | | |
| phages from | | | | | |
| feces | | | | | |
| H8 | S | T5-like | S. enterica | ? | BtuB |
| | | | serovar | | |
| | | | Enteritidis | | |
| OJ367 | ? | ? | Salmonella | ? | 45 kDa |
| | | | derby | | Omp |
| DMS3 | S | ? | Psuedomonas | ? | type IV pili |
| | | | aeruginosa | | |
| TLS | M | T-even | E. coli | TolC ? | TolC ? |
| Gifsy1, | ? | ? | S. enterica | ? | OmpC |
| Gifsy2 | | | var. | | |
| | | | Typhimurium | | |
| K139 | ? | Kappa | V. cholerae | LPS O-antigen | ? |
| | | | O1 El Tor | | |
| K20 | M | T-even | E. coli | OmpF and | OmpF and |
| | | | | LPS core | LPS core |
| phiCr30 | S | ? | C. crescentus | RsaA 130K | ? |
| | | | | protein of | |
| | | | | S-layer | |
| AP50 | Tect. | ? | Bacillus | Sap protein | ? |
| | | | anthracis | of S-layer | |
| CNRZ | M | ? | Lactobacillus | SlpH protein | ? |
| 832-B1 | | | helveticus | of S-layer | |
| SPP1 | S | SPP1 | Bacillus | glycosylated | YueB |
| | | | subtillis | poly(Gro-P) | |
| | | | | teichoic acids | |
| | | | | of the cell wall | |
| A118, P35 | S | | Lysteria | serovar-specific | ? |
| | | | monocytogenes | teichoic | |
| | | | | acids of the | |
| | | | | cell wall | |

### Host Libraries:

We used RB-TnSeq method for loss-of-function (LOF) screens to study host factors important in phage infection, and Dub-seq method for performing gain-of function (GOF) screens to study host-gene dosage and overexpression effects on phage resistance. We used E. coli BW25113 strain as host organism. The construction of E. coli BW25113 (K-12) RB-TnSeq and Dub-seq library has been presented earlier (Wetmore, et al., MBio, 6, 3, e00306-15, 2015; Mutalik et al., Nat Communications, 10, 308, 2019).

E. coli BW25113 RB-TnSeq mutant library was made up of 100,000 mutants and was created by insertion of a barcoded transposon in E. coli BW25113 (for RB-TnSeq) while GOF Dub-seq library of BW25113 was created by cloning E. coli BW25113 DNA fragments of 3 kbps into a medium copy barcoded broad-host plasmid and is made up of 30,000 member library.

For the superinfection exclusion mechanism, we combined T2, T3, T4, T5, T6, and T7 phage genomes and sheared them to 3 Kbs size fragments. These fragments were then end repaired, phosphorylated and ligated to restriction digested and dephosphorylated dual barcoded Dub-seq vector library (standard molecular biology methods). The ligated library was then transformed into cloning E. coli DH10B strain. Transformants were then collected, grown to saturation, and barcoded junctions were characterized as explained earlier (Mutalik et al., Nat Communications, 10, 308, 2019). We term this library as the phage Dub-seq library. This type of phage library is useful in not only uncovering superinfection mechanism but also to discover anti-CRISPR proteins in a large scale, cheaper and quantitative format.

### Experimental Approach

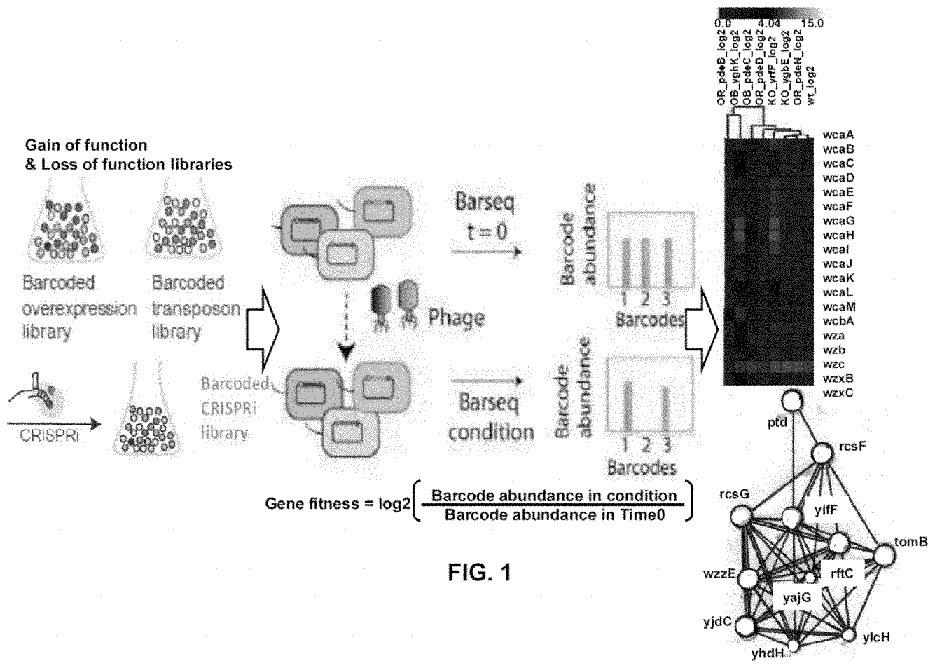

Both RB-TnSeq and Dub-seq methods rely on the use of random 20 nucleotide DNA barcodes (one barcode in the case of RB-TnSeq and two barcodes in the case of Dub-seq) and one time Illumina sequencing for characterizing initial library mapping using a TnSeq-like protocol. Both our RB-TnSeq and Dub-seq platforms use a simple, scalable barcode-sequencing assay termed Barseq and enable large-scale investigation of gene phenotypes in single-pot competitive fitness assays (FIG. 1). We performed RB-TnSeq and Dub-seq pooled fitness assays in presence of different E. coli phages in planktonic cultures at different multiplicity of infection (MOI), as well as we performed these assays on agar plates.

For both RB-TnSeq and Dub-seq experiments, we recovered a frozen aliquot of the library in LB media with antibiotic to mid-log phase, collected a cell pellet for the “start” (or time-zero sample), and used the remaining cells to inoculate an LB culture supplemented with different dilutions of a phage in SM buffer. Briefly, we used the recovered library stock and dilute it to 0.02 OD600, and then mix 350 ul of it with 350 ul of phage dilution. Then we let the culture grow at 37 C with shaking in 48 well plates in a plate reader. We periodically check the OD600 to follow the growth of surviving bacterial population. After 12 hrs of phage infection in planktonic cultures, we collected the surviving phage-resistant strains and stored at −80 C till all samples are collected.

We also repeated these fitness assays on solid media. In this step, we mix recovered 75 ul of culture of OD 600 at 0.02 and 75 ul of phage dilution. Let them stand at room temp for 5-10 minutes, and then plate mixture on a LB agar plates. We then incubated these plates at 37 C overnight and next day collected all surviving phage-resistant colonies. We hypothesized that fitness experiments on solid media might provide less stringent selection environment and far less competition for less fit survivors from highly fit resistant mutants. For the superinfection work, we repeated the phage assays by growing phage Dub-seq library in presence of different dilution of phages. We then collected survivors in both planktonic cultures and on solid plate assays.

The genomic DNA (in the case of RB-TnSeq assay) and plasmid DNA (in the case of Dub-seq assay) from these collected samples was extracted in 96-well format and strain quantification was performed using a high-throughout Barseq protocol (as explained earlier in Wetmore, et al., MBio, 6, 3, e00306-15, 2015; Mutalik et al., Nat Communications, 10, 308, 2019. We multiplexed 96 BarSeq PCR samples per lane of 50 single end read runs on Illumina sequencing as explained before (Wetmore, et al., MBio, 6, 3, e00306-15, 2015; Mutalik et al., Nat Communications, 10, 308, 2019). In each experiment, every gene has an associated fitness score, defined as the log 2 ratio of abundance of that strain in the starting pool (T0) versus the abundance after the experiment run (Tcondition). The data processing and analysis of these assays was done as previously described (Wetmore, et al., MBio. 6, 3, e00306-15, 2015; Mutalik et al., Nat Communications, 10, 308, 2019).

To formulate rationally deigned phage cocktails, we combined phages that have different target receptors and found that these cocktails are successful in overcoming bacterial resistant populations.

### Results:

To investigate host factors important in phage infection and resistance we focused on E. coli and its 6 double-stranded DNA phages for which there is a sizable amount of published work that can be used to interpret and validate the results.

### Screening for Phage Resistance Via Genome-Wide LOF Libraries

E. coli BW25113 RB-TnSeq Library:

As a demonstration of our methodology and to illustrate the scalability of our approach for genome-wide screening of host factors essential or detrimental for diverse phages, we used E. coli BW25113 RB-TnSeq library and performed competitive fitness assays in the presence of 6 different phages at different MOIs. If a particular gene product (for example, receptor) is essential for a successful phage binding and infection cycle, deletion or disruption of that gene will lead to a phage resistant strain while sensitive strains lyse. The positive fitness scores indicate that the gene(s) disrupted lead to an increase in relative fitness in presence of a particular phage and is essential for phage binding or growth. The negative fitness values indicate gene(s) disruption led to reduced relative fitness (that is mutant strains are sensitive to phage than the wild-type strain), while scores near zero indicate no fitness reduction or benefit for the mutated gene(s) under the assayed condition. In total, we performed 50 genome-wide pooled fitness assays (using E. coli RB-TnSeq library) across 6 phages at different phage dilutions. The gene fitness scores were reproducible across different phage MOI and assays systems.

We focused on the genes with positive fitness scores, as the deletion of a gene that is important for phage binding and growth is usually expected to lead to a fitness advantage in presence of phage. In total, we identified a number of positive hits for RB-TnSeq dataset with more than 50 different genes had a fitness benefit when deleted in presence of at least one phage. To confirm the validity of our approach, we looked for receptors recognized by many of the canonical phages used in this study for which there is substantial published work available. Indeed, we found highest scoring phage-specific host genes that are known to be primary receptors for a number of phages and show phage resistance when deleted (Table 1, Silva et al., FEMS Microbiology letters, 363, 2016, fnw002; Letarov and Kulikov, Biochemistry (Moscow), 82, 13, 1632-1658, 2017). These include, fadL (T2 phage), lpcA, rfaD, rfaE, waaC (check, T3 phage), ompC (T4 phage), fhuA (T5 phage), tsx (T6 phage), and rfaD, rfaE (check, T7 phage). Our data is also in agreement with gene hits identified in earlier genome-wide screens on T4, and T7 (Qimron et al., PNAS, 103, 50, 19039-19044, 2006; Rousett, et al., PLoS Genet 14, 11, e1007749; hereby incorporated by reference in their entireties). We also uncovered a number of phage resistance hits identified in disparate studies that were known to interfere or regulate phage receptors and phage growth. These high-scoring genes are known to show phage resistance either by regulating the expression of target phage receptor or because they are involved in biosynthesis of LPS, a known key recognition moiety for many phages. For example, genes involved in LPS biosynthesis (T3, T7 phage), genes involved in regulation of ompC (envZ, ompR, for T4 phage). This is the first genome-wide LOF screen applied to a number of canonical phages such as T2, T3, T5, and T6. In addition to confirming high-scoring genes that are known to be receptors for each of these phages, we find number of novel hits. We repeated these fitness experiments on LB agar plates and our results are consistent with those obtained from plaktonic growth assays.

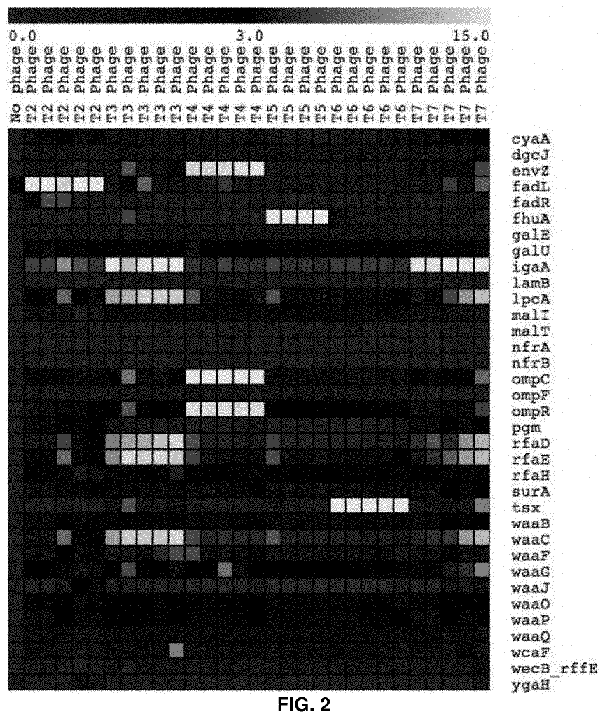

Though most gene deletions showed phage specific fitness-benefit, twelve genes had positive fitness scores in at least 2 or more phages (FIG. 2). One of which is IgaA (yrfF) gene whose deletion yields resistance to all most all phages used in this study. IgaA is an essential E. coli gene and known to regulate res phosphorylae pathway and its down regulation known to enhance colonic acid formation. Increased colonic acid formation has been predicted to mask accessibility of receptors to phages thereby leading to phage resistance phenotype (Qimron et al., PNAS, 103, 50, 19039-19044, 2006; Rousett, et al., PLoS Genet 14, 11, e1007749, 2018; hereby incorporated by reference in their entireties). Overall, our RB-TnSeq data is consistent with known literature on phage receptors and provides novel hits and insights into phage resistance across diverse dsDNA phages.

### Screening for Phage Resistance Via Genome-Wide GOF Dub-Seq Library

E. coli BW25113 Dub-seq Library

To discover gene dosage and overexpression effects of host factors on phage resistance, we used E. coli BW25113 Dub-seq library. As explained above for RB-TnSeq assays, we performed competitive fitness assays using E. coli BW25113 Dub-seq library in the presence of 6 different phages at different MOIs in planktonic cultures. Any increased dosage or overexpression of a host factor interfering with the phage binding and infection steps, may lead to phage resistant strain while sensitive strains lyse. The positive fitness scores in Dub-seq assay indicate that the gene(s) overexpression (or increased dosage) leads to an increase in relative fitness in presence of a particular phage and may be interfering with phage binding or growth. The negative fitness values indicate increased gene dosage is either toxic to the host or may sensitize cells from phage infectivity thereby reducing the relative fitness compared to the wild-type strain. The gene fitness scores near zero indicate no fitness reduction or benefit for the overexpressed or copy number amplified gene(s) under the assayed condition. In total, we performed >10 genome-wide pooled fitness assays on E. coli BW25113 strain (using E. coli BW25113 Dub-seq library) across 6 phages at different phage dilutions. Overall we identified more than 50 genes that have positive growth benefit across all phages and different genes had a fitness benefit when overexpressed in presence of at least one phage. Nearly all Dub-seq experiments had at least one gene with a positive growth effect per phage.

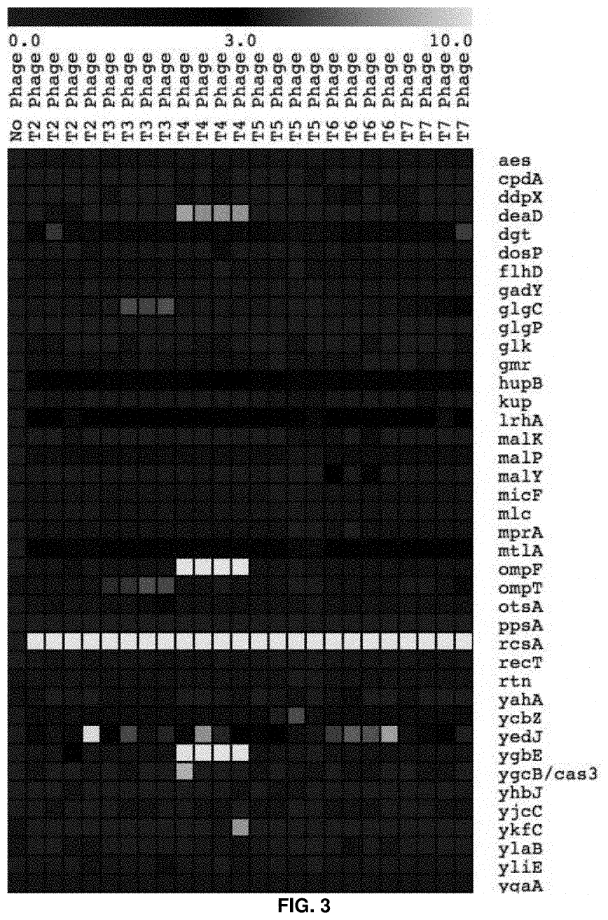

Some genes had positive fitness scores across all phages assayed in this work. Specifically, overexpression of 7 genes (rcsA, dgt, hupB, lrhA, ycbZ, mtlA and yedJ) showed resistance to all most all phages. In particular, overexpression of transcriptional activator rcsA gene known to increase colonic acid production by inducing capsule synthesis gene cluster showed highest gene score of +12 to +16 in all experiments (FIG. 3). Overexpression of rcsA is known to show resistance to T7 phage infection probably due to interference with phage receptor accessibility (Qimron et al., PNAS, 103, 50, 19039-19044, 2006). Our data is consistent with this earlier observation for T7 phage and demonstrates that formation of colonic acid capsule may be a general mechanism by which bacteria show resistance to most phages. This observation is also consistent with igaA data from K-12 RB-TnSeq.

We also identified dozens of phage-specific growth benefit E. coli K-12 genes. We identified overexpression of ygbE, ompF and deaD provide highest fitness score for T4 phage; glgC gives resistance to 186 and T3, T7; Though this is the first systematic analysis of gene dosage effect on phage resistance and we do not completely understand all of the mechanisms of resistance, many of these hits make sense in the context of known biology for some of the well-studied phages. For example, it is known that expression of outer membrane porins ompC and ompF are regulated antagonistically by ompR, and increased ompF level does reduce ompC expression. We speculate that higher copy of ompF coding and promoter region in our Dub-seq library might be titrating away ompR thereby reducing ompC expression to show T4 resistance.

### Phage Cocktail Formulation

Based on the data we obtained from both RB-TnSeq and Dub-seq, we formulated phage cocktails by combining phages that have different host targets. These combinations showed that host killing is highly efficient compared to individual phages. However overexpression of colonic acid (via overexpressing rcsA or deletion of yrfF) causes resistance to phage cocktails. These results indicated that formulation of cocktails are not always successful, and we need to gain more detailed insights about which other conditions might elevate these effects

### Superinfection Mechanim

We performed phage Dub-seq assays in E. coli BW25113 strain in presence of different phages. We found known hits among these phages we used. We also have a number of new gene hits with big scores, though we do not yet know how this supe infection mechanism is brought about.

### Discussion:

Verotoxigenic E. coli is a leading cause of millions of infections each year and causes many human deaths in developing countries (CDC.gov/ecoli). Persistence in plants, agriculture produce and water represents an important life cycle for this pathogen, and bacteriophages have been proposed as biocontrol agents. These studies (determining phage-host interaction determinants using nonpathogenic E. coli (BW25113)) are valuable in gaining understanding of pathogenic E. coli. Our exploration of these diverse E. coli strains gives us insight into how much phage resistance mechanisms vary nature and phage effectiveness as hosts vary.

Currently used approaches in studying phage-host interactions are low throughout, expensive, labor intensive and non-quantitative. Herein we presented a characterization platform to fill these technical limitations of current approaches. We extend the work to formulate cocktails based on the data we generate. Also, these studies and genetic screen easily extend to diverse biological agents such as phage like bacteriocins, peptides, antibiotics and metals.

In summary, this work is the first global survey of host genes essential for diverse phage propagation across two widely studied E. coli strains and provide a rich dataset for deeper biological insights and bioinformatic analysis. These experiments also yield a number of testable hypotheses on host specificity, resistance which are verifiable by engineering of those phage variants in genome assembly platform.

The knowledge base developed with our technology helps to develop sophisticated machine learning algorithm for predicting antimicrobial cocktails for treating microbial pathogens and manipulate microbiomes. This development of rational antimicrobial cocktail formation ultimately enables rapid deployment of solution to the hospitals and field when antibiotic resistant microbe arises.

What is claimed is:

1. A method for screening for gene function for a bacteriophage, the method comprising: (1) (a) providing one or more host organism, such as a species or strain, libraries, (b) providing randomly barcoded transposon sequencing (such as RB-TnSeq), and (c) screening for loss-of-function (LOF) mutant phenotypes; or (2) (a) providing one or more DNA barcoded overexpression strain libraries (such as Dub-seq) using DNA of the host organism and/or phage, and (b) screening for gain-of-function (GOF).

2. The method of claim 1, wherein the method comprises: (a) providing one or more host organism, such as a species or strain, libraries, (b) providing randomly barcoded transposon sequencing (such as RB-TnSeq), and (c) screening for loss-of-function (LOF) mutant phenotypes.

3. The method of claim 2, wherein the providing one or more host organism libraries comprises inserting a barcoded transposon into a host organism, such as using the method taught in Example 1, wherein the host organism(s) can be any host organism, such as any described in Table 1.

4. The method of claim 1, wherein the method comprises: (a) providing one or more DNA barcoded overexpression strain libraries (such as Dub-seq) using DNA of the host organism and/or phage, and (b) screening for gain-of-function (GOF).

5. The method of claim 1, wherein the providing one or more DNA barcoded overexpression strain libraries using DNA of the host organism and/or phage comprises cloning a partial or total host/phage genome DNA fragments into a library of barcoded vector, such as a vector that can stably reside in the host organism, wherein each resulting vector comprises a host/phage genone DNA fragment integrated into the vector, such as using the method taught in Example 1, wherein the host organism(s) can be any host organism, such as any described in Table 1.

6. The method of claim 1, wherein the providing step comprises end repairing the fragments, phosphoylating the repaired fragments, and ligating the phosphorylated repaired fragments to the vector.

7. The method of claim 1, wherein the screening step comprises transforming a phage library into cloning bacterial strain, such as an E. coli strain, collecting the transformants, growing to saturation, and characterizing barcoded junctions derived from the phage library.

8. The method of claim 4, wherein the providing one or more DNA barcoded overexpression strain libraries using DNA of the host organism and/or phage comprises shearing genomes of one or more bacteriophages inserting a barcoded transposon into a host organism, such as using the method taught in Example 1, wherein the bacteriophages(s) can be any bacteriophages(s) which correspond to a single host, such as any described in Table 1.

9. The method of claim 1, wherein there is one species of host organism and a plurality of bacteriophage species wherein each bacteriophage species is capable of infecting the host organism.

10. The method of claim 1, wherein there are a plurality of host organism species and one bacteriophage species wherein the bacteriophage species is capable of infecting each host organism species in the plurality of host organism species.

11. The method of claim 1, wherein the providing and/or screening steps are automated and/or high throughout. In some embodiments, each individual host organism and/or phage sample is provided and/or screened in a format configured for automated and/or high throughout processing and/or handling, such as a 96-well format.
